(19) United States
(12) Patent Application Publication
(10) Pub. No.: US 2018/0110794 A1
(43) Pub. Date: Apr. 26, 2018

(54) METHOD FOR IMPROVING BLADDER FUNCTION

(71) Applicant: N.V. NUTRICIA, Zoetermeer (NL)

(72) Inventor: Mattheus Cornelis DE WILDE, Utrecht (NL)

(73) Assignee: N.V. Nutricia, Zoetermeer (NL)

(21) Appl. No.: 15/558,702

(22) PCT Filed: Mar. 15, 2016
(86) PCT No.: PCT/NL2016/050184

§ 371 Date: Sep. 15, 2017

(30) Foreign Application Priority Data
Mar. 16, 2015 (NL) PCT/NL2015/050168

(51) Int. Cl. A61K 31/7072; A61K 31/20; A61P 13/10

(52) U.S. Cl. CPC A61K 31/7072; A61P 13/10; A61K 31/20

(57) ABSTRACT

The invention pertains to the use of uridine and/or an equivalent thereof and n3-PUFA in the manufacture of a product for restoring or improving bladder function in a subject, in particular in a patient suffering from spinal cord injury.

The invention is in the field of medical nutrition and more particularly relates to compositions for use in improving bladder function. In a preferred aspect, the invention pertains to improving bladder function in patients suffering from a neurological disorder.

### BACKGROUND

Impaired bladder function, bladder dysfunction or urinary incontinence is a common and serious problem, which may have a profound impact on one's life. The main cause of impaired bladder function is a damaged nerve function of the bladder, which is often associated with neurological disorders such as brain injury, spinal cord injury, sacral cord injury and peripheral nerve injury. These neurological disorders may interfere with the nerve function of the bladder. Because of the coordination required between the micturition centers, damage at any of these sites will often result in neurogenic bladder dysfunction.

Impaired bladder function with neurological cause may also be referred to as “neurogenic bladder”. Any type of lesion in the nervous system, e.g. at the cerebral level, spinal or sacral cord or the peripheral nerves, may be the cause of bladder dysfunction.

Normal bladder functioning is regulated by a synergistic cooperation of the detrusor muscle and the sphincter of the bladder. They normally have two functions, namely to collect urine and maintain continence, and to empty the bladder when necessary, without leaving residual urine behind. The detrusor muscle consists of smooth muscle fibres that can contract to facilitate the emptying of the bladder. When the wall of the bladder is stretched this will signal the parasympathetic nervous system that the bladder is full, and therefore detrusor contraction is needed to expel the excess urine. The internal and external urethral sphincters are normally contracted to prevent the bladder from emptying, and will relax to let urine pass through. The internal sphincter is autonomically controlled, while the external sphincter can be voluntarily controlled. Both the detrusor and sphincter can be in a relaxed or contracted state. Bladder dysfunction can mostly be described by 2 categories: failure to store and failure to empty. Failure to store mostly is the result of a hyperreflexive detrusor, or an areflexic sphincter. Failure to empty is mainly due to an areflexic detrusor, and a hyperreflexive sphincter.

Parasympathetically, the bladder is innervated by efferent nerve supply that originates at S2-S4 of the sacral cord, travelling towards the bladder through the use of the pelvic nerve. Parasympathetic stimulation will lead to detrusor contraction, and therefore contraction of the bladder leading to urine evacuation. Sympathetically, the efferent nerve originates at T11-L2 and travels to the bladder and urethra through the use of the hypogastric nerve. B-adrenergic receptors in the body of the bladder will cause a relaxation of the smooth muscle cells, while A-receptors in the base of bladder and urethra will cause contraction of these cells. Somatic efferents originate from S1-S4 of the sacral cord and travel through the pudendal nerve to innervate the external urethral sphincter, which can be controlled voluntarily.

Coordination of the bladder and sphincter-detrusor functioning takes place in the brain, in the pontine micturition centre, which when activated facilitates the relaxation of the urethral sphincter. This centre has a direct pathway with the sacral micturition centres to coordinate urine voiding, which requires a relaxation of the sphincter and contraction of the detrusor. The sacral micturition centre receives signals when the bladder is stretched (i.e. signalling that it is full) and triggers reflexive voiding. The pontine micturition centre matures as a child ages, so that that a person is able to control voiding, and not void reflexively. Voluntary control of urination is controlled in the medial frontal lobe and corpus callosum.

To date, no effective treatment of neurological impaired bladder function is available.

During the last decennium, uridine, choline and n-3 fatty acids such as DHA have attracted attention as active components in treating cognitive dysfunction and age-associated memory impairment (AAMI), see e.g. WO2007/089703 (Massachusetts Institute of Technology) and WO 2009/002165 (N.V. Nutricia). These compounds are rate-limiting precursors for membrane phosphatide synthesis. According to the above publications, by improving the membrane phosphatide synthesis, it is believed to improve cognitive or memory function. The effects on membrane phospholipids have been associated with enhancement in specific pre- and post-synaptic proteins.

WO 2013/066165 and WO 2013/066167 (N.V. Nutricia) disclose a product comprising (i) one or more of uridine and cytidine, or salts, phosphates, acyl derivatives or esters thereof , and (ii) a lipid fraction comprising at least one of docosahexaenoic acid (22:6; DHA), eicosapentaenoic acid (20:5; EPA) and docosapentaenoic acid (22:5; DPA), or esters thereof. Recognition and executive functions like speed of information processing, cognitive and mental flexibility, attention, scanning, and cognitive set shifting can be improved by administration of the composition, in particular in a Alzheimer's or dementia patient. WO 2012/125020 discloses a similar product for use in the prevention or treatment of neurotrauma, traumatic brain injury, cerebral palsy and spinal cord injury, focussing on neuronal survival. The same product has also been shown to enhance membrane formation and function in clinical trials with Alzheimer's disease (AD) patients (Scheltens et al. in Alzheimer's & Dementia 2010, 6, 1-10, and in J. Alzheimers Dis. 2012, 31, 225-236).

Spinal cord injury (SCI) affects a significant number of patients worldwide. Despite the increased survival rate due to advances in emergency medicine protocols, there are no neuroprotective or neuroregenerative treatments and many SCI patients suffer from lifelong motor and sensory impairment. This has a significant impact on the patients' quality of life and life expectancy and also represents a public health cost burden.

The efficacy of n-3 fatty acids in the management and improvement of spinal cord injury is reviewed by Michael-Titus et al. (in Trends in Neurosciences, 2014, 37, 30-38). Figueroa et al. (in J. Neurotrauma, 2013, 30, 853-868) describe that prophylactic n-3 PUFA administration improves functional recovery of bladder function after spinal cord injury.

### SUMMARY OF THE INVENTION

The inventors surprisingly found that a composition comprising uridine and/or an equivalent thereof and n-3 PUFA is effective in improving bladder function, in particular improving bladder function in patients suffering from, is recovering from and/or has suffered from a neurological disorder. Without being bound to a theory, it is believed that the mixture of uridine and n-3 PUFAs could improve the neurological function of the bladder and the connection between bladder and brain by supporting the regenerative processes which occur e.g. after spinal cord injury. The example shows that the composition according to the invention indeed leads to significantly improved bladder function. Bladder function is restored significantly faster upon administration of the composition according to the invention.

The present invention thus concerns a method for restoring or improving bladder function in a subject, comprising administering to the subject a composition comprising (i) uridine and/or an equivalent thereof and (ii) n-3 PUFA. The invention may also be worded as the use of (i) uridine and/or an equivalent thereof and (ii) n-3 PUFA for the manufacture of a composition for restoring or improving bladder function in a subject.

In other words, the invention concerns a composition for use in restoring or improving bladder function in a subject, said composition comprising (i) uridine and/or an equivalent thereof and (ii) n-3 PUFA. The invention also concerns a combination of (i) uridine and/or an equivalent thereof and (ii) n-3 PUFA for use in restoring or improving bladder function in a subject.

In a first preferred embodiment, the uridine and/or equivalent thereof as mentioned in the context of the invention is uridine monophosphate. In a second preferred embodiment, the n-3 PUFA is selected from EPA and/or DHA, preferably at least DHA. In a third preferred embodiment, the composition further comprises one or more of choline, B vitamin(s), said B vitamin(s) preferably comprising or being at least folic acid, more preferably at least folic acid and vitamin B6, and antioxidants. In a fourth preferred embodiment, the composition comprises at least choline. In a most preferred embodiment, the composition further comprises choline, folic acid, vitamin B6, antioxidants and phospholipids. In a fifth preferred embodiment, the subject is a patient who is suffering from, is recovering from and/or has suffered from a neurological disorder, preferably from spinal cord injury. In a sixth preferred embodiment, the composition comprises per 100 mL: (i) 400-800 mg UMP; (ii) n-3 PUFAs comprising (a) 100-500 mg EPA and (b) 900-1500 mg DHA,; (iii) 50-600 mg phospholipids; (iv) 200-600 mg choline; (v) vitamins B comprising (a) 1-5μg vitamin B12, (b) 0.5-3 mg vitamin B6 and (c) 200-600 μg folic acid; and (vi) antioxidants comprising (a) 20-60 mg vitamin E (alpha-TE), (b) 60-100 mg vitamin C, and (c) 40-80 μg selenium. In a seventh preferred embodiment, the composition is a liquid or a solid which is reconstitutable with a liquid.

### DETAILED DESCRIPTION

The present invention concerns a method for improving (impaired) bladder function in a subject, wherein the method involves administration of a composition to said subject, said composition comprising (i) uridine and/or an equivalent thereof and (ii) n-3 PUFA. Preferably, the composition according to the invention further comprises one or more selected from choline and B vitamin(s) and preferably also antioxidants, more preferably also phospholipids.

Alternatively, the invention also concerns a composition for use in improving (impaired) bladder function in a subject, said composition being characterized as above and with more detail here below. Also, the invention pertains to the use of (i) uridine and/or an equivalent thereof and (ii) n-3 PUFA in the manufacture of a composition for improving (impaired) bladder function in a subject, said composition being characterized as above and with more detail here below.

Components (i) and (ii) are present in therapeutically effective amounts.

### Composition

The method or use or composition for use according to the invention involves administration of the composition according to the invention. The composition according to the invention may be used as a pharmaceutical product or a nutritional product.

In one aspect, the composition according to the invention may be used as a pharmaceutical product comprising one or more pharmaceutically acceptable carrier materials. Such product may contain the daily dosages as defined below in one or more dosage units. The dosage unit may be in a liquid form or in a solid form, wherein in the latter case the daily dosage may be provided by one or more solid dosage units, e.g. in one or more capsules or tablets. The pharmaceutical product, preferably for enteral application, may be a solid or liquid galenical formulation. Examples of solid galenical formulations are tablets, capsules (e.g. hard or soft shell gelatine capsules), pills, sachets, powders, granules and the like which contain the active ingredients together with conventional galenical carriers. Any conventional carrier material can be utilized. The carrier material can be organic or inorganic inert carrier material suitable for oral administration. Suitable carriers include water, gelatine, gum Arabic, lactose, starch, magnesium stearate, talc, vegetable oils, and the like. Additionally, additives such as flavouring agents, preservatives, stabilizers, emulsifying agents, buffers and the like may be added in accordance with accepted practices of pharmaceutical compounding. While the individual active ingredients are suitably administered in a single composition, they may also be administered in individual dosage units.

In a preferred aspect, the composition according to the invention may be used as a nutritional product, for example as a nutritional supplement, e.g. as an additive to a normal diet, as a fortifier, to add to a normal diet, or as a complete nutrition. The nutritional product preferably comprises at least one component, preferably all components, selected from the group of fats, proteins, and carbohydrates. It is understood that a nutritional product differs from a pharmaceutical product by the presence of nutrients which provide nutrition to the subject to which the composition is administered, in particular the presence of protein, fat, digestible carbohydrates and dietary fibres. It may further contain ingredients such as minerals, vitamins, organic acids, and flavouring agents. Although the term “nutraceutical product” is often used in literature, it denotes a nutritional product with a pharmaceutical component or pharmaceutical purpose. Hence, the nutritional composition according to the invention may also be used in a nutraceutical product.

In one embodiment, the product comprises a lipid fraction and at least one of carbohydrates and proteins, wherein the lipid composition provides between 20 and 50 energy % of the food product. In one embodiment, the food product is a liquid composition containing between 0.8 and 1.4 kcal per ml.

The composition of the invention is typically an enteral composition, i.e. intended for oral administration. It is preferably administered in liquid form. Preferably, the composition comprises water in which the further components are dissolved or suspended.

The composition is thus preferably a liquid, or a solid (typically a powder or tablet, preferably a powder) which is reconstitutable with a liquid, preferably with water, to obtain a liquid composition. Dosages of components defined below may for example be in daily dose or in a concentration per 100 mL. The latter definition also applies to reconstitutable solids and should be determined after reconstitution with the liquid.

### Uridine

The present composition comprises (i) uridine and/or an equivalent thereof. Uridine equivalents are known in the art and typically include deoxyuridine (deoxyribosyl uracil), uridine phosphates (UMP, dUMP, UDP, UTP), nucleobase uracil, acylated uridine derivatives (e.g. C₁₋₆ acylated uridine) and/or esters (e.g. C₁₋₆ alkanoate ester). The composition preferably comprises a component (i) selected from uridine (ribosyl uracil), deoxyuridine (deoxyribosyl uracil), uridine phosphates (UMP, dUMP, UDP, UTP), nucleobase uracil, acylated uridine derivatives and mixtures thereof, more preferably a uridine phosphate selected from uridine monophosphate (UMP), uridine diphosphate (UDP) and uridine triphosphate (UTP). Most preferably the composition comprises UMP, as UMP is most efficiently being taken up by the body. Hence, inclusion of UMP in the present composition enables a high efficacy at the lowest dosage and/or the administration of a low volume to the subject.

Preferably at least 50 wt % of component (i) is provided by UMP, more preferably at least 75 wt %, most preferably at least 95 wt %. Doses that are to be administered are conveniently given as UMP. The amount of uridine source is thus conveniently calculated taking the molar equivalent to the UMP amount.

The present method preferably comprises the administration of uridine (the cumulative amount of uridine and equivalents thereof) in an amount of (a) 0.1 to 6 g per day, preferably 0.2 to 3 g per day, more preferably 0.4 to 2 g per day, and/or (b) 0.1 to 6 g per 100 ml (liquid) composition, preferably 0.2 to 3 g per 100 ml (liquid) composition, more preferably 0.4 to 2 g per 100 ml (liquid) composition. Based on total weight of the composition, uridine is preferably present in at least 0.1%, more preferably in at least 0.7 wt %, most preferably in at least 2.5 wt %, and/or in at most 5 wt %, more preferably in at most 3 wt %, most preferably in at most 2.5 wt %.

### Cytidine

In addition to, or instead of, uridine, the composition may also contain cytidine and/or an equivalent thereof. Cytidine equivalents are known in the art and typically include deoxycytidine (deoxyribosyl cytosine), cytidine phosphates (UMP, dUMP, UDP, UTP), nucleobase cytosine, acylated cytidine derivatives (e.g. C₁₋₆ acylated cytidine) and/or esters (e.g. C₁₋₆ alkanoate ester). In one embodiment, the composition comprises one or more selected from cytidine, cytidine phosphate (CMP, CDP, CTP, preferably CMP), citicoline (CDP-choline) may also be applied.

The present method preferably comprises the administration of cytidine (the cumulative amount of cytidine and equivalents thereof) in an amount of (i) 0.1 to 6 g per day, preferably 0.2 to 3 g per day, more preferably 0.4 to 2 g per day, and/or (ii) 0.1 to 6 g per 100 ml (liquid) composition, preferably 0.2 to 3 g per 100 ml (liquid) composition, more preferably 0.4 to 2 g per 100 ml (liquid) composition. Based on total weight of the composition, cytidine is preferably present in at least 0.1%, more preferably in at least 0.7 wt %, most preferably in at least 2.5 wt %, and/or in at most 5 wt %, more preferably in at most 3 wt %, most preferably in at most 2.5 wt %.

### n-3 PUFA

The present composition comprises (ii) n-3 polyunsaturated fatty acid (PUFA), preferably n-3 LC-PUFA. In the context of the present invention, LC-PUFAs (long-chain PUFAs) have a chain length of 18 or more carbon atoms.

Component (ii) is preferably selected from docosahexaenoic acid (22:6; DHA), eicosapentaenoic acid (20:5; EPA), docosapentaenoic acid (22:5 ω-3; DPA) and mixtures thereof, preferably at least one of DHA and EPA. Preferably the present composition contains at least DHA, more preferably DHA and EPA. EPA is converted to DPA (ω-3), increasing subsequent conversion of DPA to DHA. Hence, the present composition preferably contains a significant amount of EPA, so to further stimulate in vivo DHA formation. Component (ii), preferably DHA and/or EPA, are preferably provided as triglycerides, diglycerides, monoglycerides, free fatty acids or their salts or esters, phospholipids, lysophospholipids, glycerol ethers, lipoproteins, ceramides, glycolipids or combinations thereof. Preferably, the present composition comprises at least DHA in triglyceride form. Suitable n-3 PUFA, n-3 LC-PUFA and/or DHA sources include tuna oil, (other) fish oils, DHA rich alkyl esters, algae oil, egg yolk, or phospholipids enriched with n-3 LC-PUFA e.g. phosphatidylserine-DHA.

DHA is preferably administered in an amount of 500 to 5000 mg per day, more preferably 750 to 4000 mg per day, most preferably 1000 to 3000 mg per day. The DHA content in the composition according to the invention is preferably such that the daily DHA intake by the patient is 50-1000 mg DHA per kg total body weight of the patient, more preferably 100-800 mg/kg, more preferably 250-700 mg/kg, most preferably 350-600 mg/kg. If at all, EPA is preferably administered in an amount of 500 to 5000 mg per day, more preferably 750 to 4000 mg per day, most preferably 1000 to 3000 mg per day. These amounts of EPA apply if it is used alone or, preferably, in combination with DHA.

In case both DHA and EPA are present, the weight ratio of DHA to EPA is preferably larger than 1, more preferably 2:1 to 10:1, more preferably 3:1 to 8:1. In terms of daily dosage, the present method preferably comprises the administration of 500 to 5000 mg n-3 LC-PUFA (more preferably DHA+EPA+DPA, most preferably DHA+EPA) per day, more preferably 750 to 4000 mg per day, most preferably 1000 to 3000 mg per day.

In terms of unit dosage, the proportion of n-3 LC-PUFA (more preferably DHA+EPA+DPA, most preferably DHA+EPA) of the total fatty acids is preferably 5 to 95 wt %, more preferably 10 to 80 wt %, most preferably 15 to 70 wt %. The present composition preferably comprises 5 to 95 wt % DHA based on total fatty acids, preferably 10 to 75 w t% DHA based on total fatty acids, more preferably 10 to 60 wt % DHA based on total fatty acids. The present composition preferably comprises 5 to 95 wt % EPA based on total fatty acids, preferably 10 to 75 wt % EPA, most preferably 15 to 60 wt %, based on total fatty acids.

Based on total weight of the composition, n-3 PUFA is preferably present in at least 0.1%, more preferably in at least 0.8 wt %, most preferably in at least 1.4 wt %, and/or in at most 5 wt %, more preferably in at most 3 wt %, most preferably in at most 2.5 wt %. Based on total weight of the composition, DHA is preferably present in 0.25-5 wt %, more preferably in 0.5-2.4 wt %, most preferably in 0.9-1.5 wt %. Based on total weight of the composition, EPA is preferably present in 0.05-2.5 wt %, more preferably in 0.2-1.0 wt %, most preferably in 0.35-0.8 wt %.

The above-mentioned ratios and amounts take into account and optimise several aspects, including taste (too high LC-PUFA levels reduce taste, resulting in a reduced compliance), balance between DHA and precursors thereof to ensure optimal effectiveness while maintaining low-volume formulations.

### Further Lipid Components

Next to n-3 PUFAs, the composition preferably comprises further lipids, such as n-6 PUFAs or n-6 LC-PUFAs (such as alpha-linolenic acid (ALA), linoleic acid (LA)) and phospholipids.

It is preferred that the ALA content of the composition is maintained at low levels. As the composition is especially beneficial for spinal cord injury patients, excess supply of highly unsaturated fatty acids is believed to result in increased risk of further damage to injury tissue, due to the effect of peroxidized PUFAs, even though it has been observed that in vivo supply of ALA is neuroprotective in neurotrauma (King et al. J. Neurosci. (26) 17:4672-4680). The ALA concentration is preferably maintained at levels less than 2.0 wt %, more preferably below 1.5 wt %, particularly below 1.0 wt %, based on the weight of all fatty acids.

LA concentrations can be maintained at normal levels, i.e. between 20 to 30 wt %, based on the weight of all fatty acids, although in one embodiment the LA concentration is also significantly reduced to an amount of below 15 wt % and even less than 10 wt %, based on total fatty acids. The LA concentrations are preferably at least 1 wt % of the fatty acids.

In one embodiment, the weight ratio n-3 PUFAs : n-6 PUFAs in the composition according to the invention is preferably in the range of 0.3 to 7, preferably in the range of 1.4:1 to 5.9:1, more preferably in the range of 3:1 to 5.5:1, most preferably in the range of 3:1 to 5:1, in particular less than 5:1. The amount of n-6 LC-PUFAs is preferably less than 50 wt %, preferably in the range of 5 to 40 wt %, more preferably 8 to 30 wt %, based on total weight of the fatty acids in the composition.

The present composition may further comprise phospholipids. Preferably, one or more phospholipid(s) is/are present in the composition according to the invention. The one or more phospholipid(s) is/are selected from the group consisting of phosphatidic acid (PA), phosphatidylethanolamine (PE), phosphatidylcholine (PC), phosphatidylserine (PS) and phosphoinositides (PI). The present composition preferably comprises at least one phospholipid in an amount of 0.01 to 1 gram per 100 ml, more preferably between 0.05 and 0.5 gram per 100 ml, most preferably 80 to 600 mg per 100 ml. The at least one phospholipid is preferably provided by lecithin.

### Further Components

The composition according to the invention may comprise further components, for example one or more selected from choline and B vitamin(s), preferably both, and more preferably also antioxidants. The presence of one or more of, preferably all of, choline, B vitamin(s), especially folic acid and vitamin B6, and antioxidants, especially vitamin C and/or E, is preferred, since spinal cord injury has been suggested to lead to nutritional deficiencies in these components (Fraser 2014). As such, the presence of choline, B vitamin(s), especially vitamin B12, and antioxidants, especially selenium, vitamin C and/or E, may contribute to the general health of patients suffering from spinal cord injury.

### Choline

The present composition preferably comprises choline. Choline may be present as such, or as choline equivalent in the form of e.g. salt or ester form, or any combination thereof. The choline salt is preferably selected from choline chloride, choline bitartrate, or choline stearate. The choline ester is preferably selected from a phosphatidylcholine and lyso-phosphatidyl choline.

The present method preferably comprises the administration of more than 50 mg choline per day, preferably 80 to 3000 mg choline per day, more preferably 100 to 2000 mg choline per day, most preferably 150 to 1000 mg choline per day. The present composition preferably comprises 80 mg to 3000 gram choline per 100 ml of the liquid composition, preferably 100 mg to 2000 mg choline per 100 ml, preferably 200 to 1000 mg choline per 100 ml composition, most preferably 200 mg to 600 mg choline per 100 ml. The above numbers are based on choline, the amounts of choline equivalents or sources can be calculated taking the molar equivalent to choline into account.

### B Vitamins

The present composition may further comprise one or more B vitamin(s) . The vitamin B is selected from the group of vitamin B1 (thiamine), vitamin B2 (riboflavin), vitamin B3 (niacin or niacinamide), vitamin B5 (pantothenic acid), vitamin B6 (pyridoxine, pyridoxal, or pyridoxamine, or pyridoxine hydrochloride), vitamin B7 (biotin), vitamin B9 (folic acid or folate), and vitamin B12 (various cobalamins). Functional equivalents are encompassed within these terms. The term “vitamin B12” incorporates all cobalamin equivalents known in the art. Preferably, B vitamins in the context of the invention comprises at least one, more preferably at least two, selected from the group of vitamin B6, vitamin B12 and vitamin B9. More preferably the composition comprises at least vitamin B6 and/or B9, most preferably vitamin B6, B9 and B12.

The vitamin B is to be administered in an effective dose, which dose depends on the type of vitamin B used. As a rule of thumb, a suitable minimum or a maximum dose may be chosen based on known dietary recommendations, for instance as recommended by Institute of Medicine (TOM) of the U.S. National Academy of Sciences or by Scientific Committee on Food (a scientific committee of the EU), the information disclosed herein and optionally a limited amount of routine testing. A minimum dose may be based on the estimated average requirement (EAR), although a lower dose may already be effective. A maximum dose usually does not exceed the tolerable upper intake levels (UL), as recommended by IOM.

When present in the composition according to the invention, vitamin B6 is usually present in an amount to provide a daily dosage in the range of 0.1 to 100 mg, in particular in the range of 0.5 to 25 mg, more in particular in the range of 0.5 to 5 mg. The present composition preferably comprises 0.1 to 100 mg vitamin B6 per 100 g (liquid) product, more preferably 0.5 to 5 mg vitamin B6 per 100 g (liquid) product, more preferably 0.5 to 5 mg vitamin B6 per 100 g (liquid) product.

When present in the nutritional composition or medicament, the vitamin B9 is usually present in an amount to provide a daily dosage in the range of 50 to 5000 μg, in particular in the range of 100 to 1000 μg, more in particular in the range of 200 to 800 μg. The present composition preferably comprises 50 to 5000 μg vitamin B9 per 100 g (liquid) product, more preferably 100 to 1000 μg vitamin B9 per 100 g (liquid) product, more preferably 200 to 800 μg folic acid per 100 g (liquid) product. Vitamin B9 may be present as folate, which includes folic acid, folinic acid, methylated, methenylated and formylated forms of folates, their salts or esters (e.g. C1-6 alkyl ester), as well as their derivatives with one or more glutamic acid, and all in either reduced or oxidized form. Preferably, vitamin B9 is provided as folic acid.

When present in the composition according to the invention, the vitamin B12 is usually present in an amount to provide a daily dosage in the range of 0.5 to 100 μg, in particular in the range of 1 to 10 μg, more in particular in the range of 1.5 to 5 μg. The present composition preferably comprises 0.5 to 100 μg vitamin B12 per 100 g (liquid) product, more preferably 1 to 10 μg vitamin B12 per 100 g (liquid) product, more preferably 1.5 to 5 μg vitamin B12 per 100 g (liquid) product.

### Antioxidants

The present composition may further comprise antioxidants, preferably selected from vitamin C, vitamin E and selenium. It is especially preferred that the composition comprises both vitamin C and vitamin E, most preferably the composition according to the invention comprises vitamin C, vitamin E and selenium. Antioxidants are preferably included in the composition according to the invention, as they may prevent oxidative damage to the injury site resulting from dietary PUFAs.

Vitamin C includes functional equivalents thereof, and may be present in an amount to provide a daily dosage in the range of 20 to 2000 mg, in particular in the range of 30 to 500 mg, more in particular in the range of 75 to 150 mg. In one embodiment, vitamin C is present in an amount in the range of 20 to 2000 mg, in particular in the range of 30 to 500 mg, more in particular in the range of 75 to 150 mg per 100 ml of the composition.

Vitamin E refers to compounds having vitamin E activity as known in the art, typically tocopherol and/or an equivalent thereof. Vitamin E may be present in an amount to provide a daily dosage in the range of 10 to 300 mg, in particular in the range of 30 to 200 mg, more in particular in the range of 35 to 100 mg. Such amounts of vitamin E prevent oxidative damage to the injury site resulting from dietary PUFA present in the composition according to the invention. In one embodiment, tocopherol and/or equivalent is present in an amount in the range of 10 to 300 mg, in particular in the range of 30 to 200 mg, more in particular in the range of 35 to 100 mg per 100 ml of the composition. The term “tocopherol and/or an equivalent thereof”, as used in this description, comprises tocopherols (e.g. alpha- and gamma-), tocotrienols, pharmaceutical and/or nutritional acceptable derivatives thereof and any combination thereof. The above numbers are based on alpha-tocopherol equivalents (alpha-TE), as recognized in the art.

The present composition preferably contains selenium. The antioxidant activity of selenium advantageously prevents and/or inhibits damages to the brain areas. Preferably the composition comprises 0.01 and 5 mg selenium per 100 ml liquid product, preferably 0.02 and 0.1 mg selenium per 100 ml liquid product. The amount of selenium administered per day is preferably more than 0.01 mg, more preferably 0.01 to 0.5 mg.

In view of the above, the composition according to the invention preferably comprises uridine and/or an equivalent thereof, the n-3 LC-PUFAs DHA and EPA, phospholipids, choline, folic acid, vitamin B12 and vitamin B6, in any of the aforementioned forms, equivalents or derivatives. The composition preferably comprises uridine and/or UMP, the n-3 LC-PUFAs DHA and EPA, phospholipids, choline, folic acid, vitamin B12, vitamin B6, vitamin C, vitamin E and selenium, in any of the aforementioned forms, equivalents or derivatives.

In an especially preferred embodiment, the composition according to the invention comprises per daily dosage or per 125 ml of liquid:
(i) 400-1000 mg, preferably 500-700 mg, more preferably about 625 mg UMP,
(ii-a) 100-500 mg, preferably 200-400 mg, more preferably about 300 mg EPA,
(ii-b) 900-2000 mg, preferably 950-1300 mg, more preferably about 1200 mg DHA,
(iii) 50-600 mg, preferably 60-200 mg, more preferably about 106 mg phospholipids,
(iv) 200-800 mg, preferably 300-500 mg, more preferably about 400 mg choline,
(v-a) 1-5 μg, preferably 2-4 μg, more preferably about 3 μg vitamin B12,
(v-b) 0.5-3 mg, preferably 0.5-2 mg, more preferably about 1 mg vitamin B6,
(v-c) 200-800 μg, preferably 300-500 μg, more preferably about 400 μg folic acid.
(vi-a) 20-80 mg, preferably 30-50 mg, more preferably about 40 mg vitamin E (alpha-tocopherol equivalents (alpha-TE)),
(vi-b) 60-150 mg, preferably 60-90 mg, more preferably about 80 mg vitamin C, and
(vi-c) 40-100 μg, preferably 45-65 μg, more preferably about 60 μg selenium.

In an especially preferred embodiment, the composition according to the invention comprises per daily dosage or per 125 ml of liquid:
(i) 600-1500 mg, preferably 700-1050 mg, more preferably about 940 mg UMP,
(ii-a) 150-750 mg, preferably 320-600 mg, more preferably about 450 mg EPA,
(ii-b) 1000-3000 mg, preferably 1400-2000 mg, more preferably about 1800 mg DHA,
(iii) 75-900 mg, preferably 110-300 mg, more preferably about 160 mg phospholipids,
(iv) 300-120 mg, preferably 450-750 mg, more preferably about 600 mg choline,
(v-a) 1.5-7.5 μg, preferably 4-6 μg, more preferably about 4.5 μg vitamin B12,
(v-b) 0.75-4.5 mg, preferably 1.2-3 mg, more preferably about 1.5 mg vitamin B6,
(v-c) 300-1200 μg, preferably 450-750 μg, more preferably about 600 μg folic acid.
(vi-a) 30-120 mg, preferably 45-75 mg, more preferably about 60 mg vitamin E (alpha-tocopherol equivalents (alpha-TE)),
(vi-b) 70-225 mg, preferably 90-135 mg, more preferably about 120 mg vitamin C, and
(vi-c) 50-150 μg, preferably 65-100 μg, more preferably about 80 μg selenium.

### Application

The composition according to the invention is for restoring or improving (impaired) bladder function or improving recovery of bladder function in a subject. The present use may also be worded as stimulating (recovery of) bladder function or bladder control, improving recovery of bladder function or bladder control, improving autonomic bladder function, treatment and/or prevention of urinary incontinence, treatment and/or prevention of leaky bladder. In the context of the invention, “prevention” may also be referred to as “reducing the risk or occurrence of”. In the context of the present invention, the impaired bladder function may take any form, such as incontinence (e.g. urge incontinence, overflow incontinence), spastic bladder, urinary retention, hypocontractile bladder, frequent urination, nocturia, overactive bladder, decrease in or loss of (full) bladder sensation, increase residual urine after voiding.

The impaired bladder function is preferably associated with or caused by a neurological disorder. In a preferred embodiment, the impaired bladder function is associated with or caused by spinal cord injury or traumatic brain injury, most preferably by spinal cord injury. In other words, the composition according to the invention is for improving (impaired) bladder function after spinal cord injury or traumatic brain injury, most preferably after spinal cord injury. In other words, the composition according to the invention is for improving (impaired) bladder function in a patient who is suffering from, recovering from and/or has suffered from a neurological disorder, in particular from spinal cord injury or traumatic brain injury, most preferably from spinal cord injury.

In a preferred embodiment, the subject is a patient suffering from, is recovering from and/or has suffered from a neurological disorder, preferably the patient is suffering from a neurological disorder. In the context of this embodiment, the use may also be referred to as “treatment and/or prevention of neurogenic bladder dysfunction” or “treatment and/or prevention of neurogenic bladder”. The neurological disorder may be any kind of injury in the nervous system of the patient, such as brain injury, spinal cord injury, sacral cord injury and peripheral nerve injury. Because of the coordination required between the micturition centers, damage at any of these sites will often result in neurogenic bladder dysfunction. In an especially preferred embodiment, the patient is suffering from, is recovering from and/or has suffered from spinal cord injury, preferably the patient is suffering from spinal cord injury.

Brain injury (lesion site located at the pons or higher) may lead to impaired or destroyed Pontine Micturition Center control, thereby causing a loss of voiding control. Often primitive voiding will be intact, and a person will become urge incontinent. Impaired bladder control caused by brain lesions typically takes the form of urge incontinence and spastic bladder. Any type of brain injury may cause impaired bladder function, the brain injury is preferably selected from stroke, brain tumour, traumatic brain injury, Parkinson's disease, hydrocephalus, cerebral palsy and Shy-Drager syndrome.

Spinal cord injury (lesion site between pons and sacral spinal cord) typically cause (complete or partial) shutdown of the central nervous system, and reactivation thereof may lead to hyperstimulation of affected organs and spasticity. Damage at the level of the spinal cord above the sacral cord will result in the loss of information relay between the pontine micturition center and the sacral micturition center, often resulting in the same type of incontinence as suprapontine lesions will result in. However, during the initial phase after a spinal cord injury the patient will often be in a state of spinal shock, which results in a reflexive nervous system shutdown and an areflexive detrusor. Therefore for the first few weeks a person is unable to void, which can be a life-threatening condition if not dealt with. After 6-8 weeks a person will be able to void reflexively since the connection with the sacral micturition center is still intact. This results in urge incontinence. Furthermore, reactivation of the nervous system can lead to hyperstimulation and spasticity of the affected organs, leading to a spastic bladder and dyssynergia between sphincter and detrusor. Impaired bladder control caused by spinal cord injury typically takes the form of urge incontinence and spastic bladder. Any type of spinal cord injury may cause impaired bladder function, the spinal cord injury is preferably selected from traumatic spinal cord injury, paraplegia, quadriplegia, Multiple Sclerosis and myelomeningocele.

Sacral cord injury (lesion site in sacral spinal cord and/or nerve roots) may cause difficulty or even inability to sense when the bladder is full (sensory neurogenic bladder) and difficulty in eliminating urine when feeling a full bladder (motor neurogenic bladder). Damage at the level of the sacral cord typically results in an inability to sense when the bladder wall is stretched. There is no voluntarily or reflexive voiding, leading to an inability to contract the bladder. Therefore a person will be unable to urinate, unless there is overflow incontinence (when the pressure inside the bladder is higher that the pressure the sphincter can maintain to remain continence). Impaired bladder control caused by sacral cord injury typically takes the form of overflow incontinence and urinary retention. Any type of sacral cord injury may cause impaired bladder function, the sacral cord injury is preferably selected from sacral cord tumour, herniated disc, crushed pelvis, lumbar laminectomy, radical hysterectomy, abdominoperineal resection and Tethered Cord Syndrome.

Peripheral nerve injury (lesion site in peripheral nerves) may cause damage to or even destroy the nerves to the bladder, which in turn can lead to the loss of sensation of bladder filling. Damage at the level of the peripheral nerves that innervate the bladder will result in no signals being able to be received to and from the bladder. There is no longer a sensation of the bladder filling, or the ability to reflexively or voluntarily void. Typically, the patient is not able to contract the detrusor (motor neurogenic bladder). Impaired bladder control caused by peripheral nerve injury typically takes the form of overflow incontinence, urinary retention and a hypocontractile bladder. Any type of peripheral nerve injury may cause impaired bladder function, the peripheral nerve injury is preferably selected from diabetes mellitus, diabetic cystopathy AIDS, poliomyelitis, Guillain-Barré syndrome, herpes, herpes zoster, pernicious anemia and neurosyphilis (tabes dorsalis).

In a preferred embodiment, the neurological disorder is selected from paraplegia, quadriplegia, Multiple Sclerosis and myelomeningocele, Parkinson's disease, stroke, traumatic spinal cord injury or traumatic brain injury. In a preferred embodiment, the neurological disorders is a traumatic injury, preferably traumatic brain injury or traumatic spinal cord injury, more preferably traumatic spinal cord injury. Especially spinal cord injury patients benefit from the composition according to the invention, as complete recovery from spinal cord injury is mostly impossible and symptoms persist throughout the entire lifespan. Discomfort from impaired ladder control is greatest for these patients.

The compositions as described above can be used as a nutritional therapy, nutritional support, as a medical food, as a food for special medical purposes or as a nutritional supplement. Such product can be consumed at one, two or three servings of 50-250 mL per day. typically of 125 mL per day during recovery and/or rehabilitation in the context of the impairments according to the invention. Preferred daily dosages are in the range of 100 to 500 mL, more preferably 125 to 375 mL, most preferably 200 to 300 mL.

Preferably, the composition is enterally administered. Administration occurs preferably at least one time per day, although alternative dosimen regimes can be calculated from these numbers.

### EXAMPLES

### Example 1

Female adult Sprague-Dawley rats (˜250g) were used in this project. The spinal cord of all animals was injured at thoracic level T12 (T12) using a static compression model (Nystrom et al., Acta Neurologica Scandinavica, 1998, 78, 460-6; Huang et al., European Journal of Neuroscience, 2007, 23, 273-8). After surgery, the rats were monitored regularly for any adverse effects, and weighed daily during the first two weeks post-injury, and then twice weekly thereafter. During the first week post-surgery, bladders were checked twice daily and were expressed manually when needed, and then once daily thereafter until the voiding reflex was re-established.

Rats were randomized to control and inventive diet groups before surgery. Rats in the control group received a regular AIN-93 M based rat chow (N=9), whereas rats in the inventive group were fed an inventive diet containing the same rat chow supplemented with a daily dose of 450 mg/kg (N=9) for 4 weeks. Both diets were isoenergetic and fulfilled all basic dietary requirements. They contained the standard vitamin mix (AIN-93-VX) and mineral mix (AIN-93-MX). The composition of the two diets differed with regard to the fat blends used, as well as a number of supplemented nutrients, included choline, B-vitamins, antioxidants, uridine monophosphate (UMP), and lecithin. The detailed composition of the diets is presented in Tables 1 and 2. To prevent lipid oxidation, all diets were stored at −20° C. until use. The diets were presented to the animals as hard pellets. All rats from both treatment groups received fresh diet pellets daily. Dietary treatment started immediately after recovery from surgery, once the rats were put back into their home cage, and a maximum of 4 rats were housed per cage. The amount of food eaten in each cage was monitored daily. Mean daily intakes were similar in all treatment groups.

TABLE 1
Diet composition (in g per 100 g of the composition)

| | Control diet | Inventive diet |
|---|---|---|
| corn starch | 35.6 | 31.3 |
| casein (>85% protein) | 14.0 | 14.0 |
| corn dextrine | 15.5 | 15.5 |
| sucrose | 10.0 | 10.0 |
| dextrose | 10.0 | 10.0 |
| fibre | 5.0 | 5.0 |
| mineral mix (AIN-93M-MX) | 3.5 | 3.5 |
| vitamin mix (AIN-93-VX) | 1.0 | 1.0 |
| oil blend | | |
| soy oil | 1.9 | 0.0 |
| coconut oil | 0.9 | 0.1 |
| corn oil | 2.2 | 0.1 |
| DHA25 oil (Nippon Suisan | 0.0 | 4.5 |
| Kaisha, LTD Tokyo, Japan) | | |
| EPA 28/12 (Biosearch Life, | 0.0 | 0.3 |
| Granada, Spain) | | |
| UMP disodium (24% H2O) | 0.0 | 1.5 |
| dl-α-tocopheryl acetate (500 IU/g) | 0.0 | 0.705 |
| Pyridoxine-HCL | 0.0 | 0.0053 |
| Folic acid (90%) | 0.0 | 0.0011 |
| Cyanocobalamin (0.1% in mannitol) | 0.0 | 0.0065 |
| Choline bitartrate | 0.25 | 0.25 |
| Choline chloride | 0.0 | 0.67 |
| Sodium selenite | 0.0 | 0.00036 |
| Tert-butylhydroquinone | 0.0008 | 0.0008 |
| L-cystine | 0.18 | 0.18 |

TABLE 2
Fatty acid profile (in g per 100 g of the composition)

| | Control diet | Inventive diet |
|---|---|---|
| C-18:1n9 | 1.041 | 0.656 |
| C-18:2n6 (LA) | 2.181 | 0.158 |
| C-20:4n6 (AA) | 0.00 | 0.092 |
| C-18:3n3 (ALA) | 0.107 | 0.038 |
| C-20:5n3 (EPA) | 0.00 | 0.433 |
| C-22:6n3 (DHA) | 0.00 | 1.117 |
| total n-6 | 2.181 | 0.316 |
| total n-3 | 0.107 | 1.663 |
| n-3/n-6 | 0.049 | 5.265 |

Statistical tests were performed using GraphPad Prism version 6 (GraphPad Software Inc., San Diego, Calif., USA). Data sets were analyzed with Student's t tests or with two-way repeated-measures ANOVAs followed by post hoc analysis using the Bonferroni's post hoc comparisons test, where appropriate. All data are given as mean±S.E.M. P<0.05 was considered statistically significant.

Comparison of the group of rats that were fed the inventive diet with that that were fed the standard maintenance diet showed that the ability for bladder voiding recovered significantly quicker in the rats treated with the inventive diet (days to bladder recovery (control diet)=8.8±1.8; (inventive diet)=3.9±1.1, P<0.05). In the inventive diet group, four rats out of seven had already recovered full control of their bladder by post-surgery day 2, and all rats had recovered bladder function by day 8. In the control group, only two rats out of 6 had recovered bladder voiding by post-surgery day 10, and all rats had recovered bladder function by day 12.

### Example 2

A liquid composition according to the invention, comprising per 125 mL serving:

| | | | |
|---|---|---|---|
| 4.9 | g fat | 40 | mg vitamin E (alpha-TE) |
| 300 | mg EPA | 80 | mg vitamin C |
| 1200 | mg DHA | 60 | μg selenium |
| 106 | mg phospholipids | 3 | μg vitamin B12 |
| 400 | mg choline | 1 | mg vitamin B6 |
| 625 | mg UMP | 400 | μg folic acid |

1-13. (canceled)

14. A method for restoring or improving bladder function in a subject, comprising administering to the subject a composition comprising:
(i) uridine and/or an equivalent thereof and
(ii) n-3 PUFA.

15. The method according to claim 14, wherein the subject is suffering from, is recovering from and/or has suffered from a neurological disorder.

16. The method according to claim 15, wherein the neurological disorder is from spinal cord injury.

17. The method according to claim 14, wherein the n-3 PUFA is selected from EPA and/or DHA.

18. The method according to claim 17, wherein 500-5000 mg DHA is administered per day.

19. The method according to claim 14, wherein the composition further comprises choline.

20. The method according to claim 14, wherein the composition further comprises B vitamin(s).

21. The method according to claim 14, wherein the composition further comprises phospholipids, choline, B vitamin(s) and antioxidants.

22. The method according to claim 21, wherein the composition comprises choline, vitamin B6 and folic acid.

23. The method according to claim 14, wherein the uridine and/or equivalent thereof is uridine monophosphate.

24. The method according to claim 14, wherein the composition comprises per 125 mL or per daily dosage:
(i) 400-800 mg UMP;
(ii) n-3 PUFAs comprising (a) 100-500 mg EPA and (b) 900-1500 mg DHA;
(iii) 50-600 mg phospholipids;
(iv) 200-600 mg choline;
(v) vitamins B comprising:
(a) 1-5 μg vitamin B12,
(b) 0.5-3 mg vitamin B6 and
(c) 200-600 μg folic acid; and
(vi) antioxidants comprising
(a) 20-60 mg vitamin E (alpha-TE),
(b) 60-100 mg vitamin C, and
(c) 40-80 μg selenium.

25. The method according to claim 14, wherein the composition is a liquid, or a solid which is reconstitutable with a liquid.
